(19) United States
(12) Patent Application Publication
(10) Pub. No.: US 2024/0149073 A1
(43) Pub. Date: May 9, 2024

(54) SKIN TREATMENT DEVICE

(71) Applicant: IPULSE LIMITED, Swansea (GB)

(72) Inventor: Stuart Jones, Swansea (GB)

(21) Appl. No.: 18/282,125

(22) PCT Filed: Mar. 15, 2022
(86) PCT No.: PCT/GB2022/050654

§ 371 Date: Sep. 14, 2023

(30) Foreign Application Priority Data
Mar. 15, 2021 (GB) 2103561.3

(51) Int. Cl. A61N 5/06

(52) U.S. Cl. CPC A61N 5/0616; A61N 2005/0626; A61N 2005/0632; A61N 2005/0644; A61N 2005/0664

(57) ABSTRACT

A skin treatment device, typically but not exclusively an IPL device, has a housing, a light source for discharging light energy pulses, a control system for controlling discharge of the light source and a housing output window in the housing for transmission of light energy pulses emitted by the light source to external of the housing onto a skin treatment area. One or more sensors are disposed in the housing adjacent to the output window to provide sensing zones. The control system is arranged to receive one or more sensor outputs and based on these outputs control operation of the device. A head is releasably engaged with the housing and has a shield portion and a head window portion where in an engaged configuration the shield portion partially shields the output window to reduce the skin treatment area and leave one or more of the sensing zones exposed.

### CROSS-REFERENCE TO RELATED APPLICATIONS

This application is the U.S. National Stage of PCT/GB2022/050654 filed on Mar. 15, 2022, which claims priority to United Kingdom Patent Application 2103561.3 filed on Mar. 15, 2021, the entire content of both are incorporated herein by reference in their entirety.

### FIELD OF THE INVENTION

The present invention relates to a skin treatment device, preferably a skin treatment device for treating unwanted hair, and preferably comprising an Intense Pulsed Light (IPL) device.

### BACKGROUND OF THE INVENTION

Skin treatment devices are known in the art for treatment of, for example, cosmetic applications such as hair depilation, minimisation of skin blemishes or skin rejuvenation, as well as dermatological treatment of skin conditions such as acne or rosacea. The skin is exposed to dosages of radiation from a light source such as a flashlamp or laser where the radiation is targeted to the skin and the energy intensity and pulse duration is controlled. In hair depilation, the radiation source is targeted to cause heating of the hair root causing the hair root to die.

Safety of skin treatment devices is paramount and is particularly important for devices designed for home use. As such, safety features are implemented so that the device will not emit radiation unless the device is in contact with a user's skin to minimise stray optical radiation from the device in operation. This is typically achieved through the provision of multiple sensors adjacent each side of the output window (for example above, below and to either side of a rectangular output window) in the head of the device where a surface must be detected by each sensor as a requirement for radiation to be emitted. If one sensor does not measure a threshold value, then it is determined by the control system of the device that there is no skin contact and firing is prevented. This is to prevent the device firing when good contact with the skin is not achieved with the associated risk of the emission of potentially harmful levels of stray radiation.

Whilst the safety features of the device limit stray radiation, there is an impact on useability. For body areas with a large flat surface, usability is good as a user can readily position the device such that all of the sensors contact the body thus allowing emission of radiation and ease of treatment. However, on more angular or bony areas of the body it is more difficult to orient the head so that all the sensors are in body contact and emission of radiation is prevented. This causes frustration for the user, and the efficacy of the treatment is reduced as difficult to treat body areas are missed. Usability is therefore decreased.

Aspects of the present invention address these problems or at least provide a useful alternative.

According to a first aspect of the present invention there is a skin treatment device comprising:
a housing;
a light source housed within the housing for discharging light energy pulses;
a control system for controlling discharge of the light source;
a housing output window in the housing for transmission of the light energy pulses emitted by the light source to external of the housing onto a skin treatment area;
one or more sensors disposed in the housing adjacent the housing output window to provide a plurality of sensing zones, where the control system is arranged to receive one or more sensor outputs from the one or more sensors and based on the one or more sensor outputs control operation of the device;
a head arranged to be releasably engaged with the housing and having a shield portion and a head window portion, where in an engaged configuration the shield portion partially shields the output window to reduce the skin treatment area and leaves one or more of the plurality of sensing zones exposed.

### SUMMARY OF THE INVENTION

This invention therefore provides a simple and effective solution to a problem of how to reduce the stray optical radiation when treating certain body areas such as bony areas in a cost effective and simple manner and still maintain useability. Accordingly, in the engaged configuration the output window is shielded and therefore the skin treatment area is reduced. One or more of the sensing zones remain exposed and thus the one or more sensors are operable to provide sensor outputs to control operation of the device. This provides a simple and effective device where the head does not require the addition of sensors or complex electronics but means that the device can operate safely with stray optical radiation minimised. The skin treatment device is beneficially operable both with and without the head engaged with the housing.

Controlling operation of the device may comprise one or both of determining whether or not the flashlamp can emit a pulse and determining properties of that pulse (e.g. fluence).

The head preferably comprises a recessed portion arranged to receive a user body portion. It will be appreciated that the recessed portion has a skin contact surface. Through the provision of a recessed portion, difficult to treat body geometry can be received and stray light minimised. This body geometry may, for example, be bony areas such as the skin over a shin bone.

The recessed portion is preferably concave, and preferably disposed between opposing shoulders. The opposing shoulders are preferably mirrored on either sides of the head output window. The opposing shoulders preferably extend generally parallel to one another. The shoulders preferably extend generally linearly in a direction parallel to the height of the output window.

The curvature of the concave recessed portion is preferably defined at least in part by a curvature radius. A curvature radius reflects the shape of typical body areas on which the head is particularly suitable. The curvature radius itself may depend on the body area to be treated, and different heads with size parameters may be utilised for different body areas. As an example, the curvature radius may have a numerical value defined by the effective width of the output window as shielded plus a predetermined value, wherein the predetermined value may be 20-60 mm, even more preferably 30-50 mm and even more preferably 40 mm. It will be appreciated that the entirety of the concave recessed portion may not comprise a curvature radius, and the curvature radius may extend from the shoulders towards the head output window with an intermediate portion that may for example be more linear or may comprise a lesser curvature.

There are significant advantages associated with the recessed portion, particularly a recessed portion having a curvature radius with the defined by the width of the output window plus a predetermined value. By providing such a significant curvature relative to the width of the output window, stray light is minimised, whilst also meaning that it is unnecessary for the provision of any sensors extending width wise beyond the first and second ends of the output window.

The head is beneficially rigid. Accordingly, under normal operation the head, particularly the shoulder portions, do not deform to conform to a user body portion.

The window portion of the head preferably comprises an opening. There is thus no physical window in the opening.

The housing output window is preferably defined by a width and a height, where the width is greater than the height and where the shield portion shields the output window to reduce the width of the light transmission area in the engaged configuration.

The head output window is preferably defined by a width and a height aligned with the width and height respectively of the housing output window in the engaged configuration, where the width of the head output window is less than the height of the head output window.

The height of the head output window is preferably greater than the height of the housing output window.

The skin treatment apparatus preferably comprises a plurality of sensors. The plurality of sensors may comprise proximity sensors (such as capacitive sensors) and/or optical proximity sensors depending on the specific functionality required. However, in any event it is beneficial that the sensors can be used to determine proximity of a surface (skin) to the sensor. If an optical proximity sensor is utilised, additional functionality may be provided such as the ability to use the sensor output (reflectance) in determination of the skin tone and therefore control the energy output by the light source dependent upon the skin tone.

The one or more sensors preferably comprises at least first and second sensors disposed in the housing on diametrically opposing first and second sides of the housing output window, and where the first and second sensors remain exposed in the engaged configuration.

The first and second sensors are preferably disposed on first and second sides above and below the housing output window. The first and second sensors are preferably disposed in the recessed portion of the head, preferably such that at least a portion of the first and second sensors are positioned at the deepest part of the recessed portion.

The shape of the output window is generally rectangular in the illustrative embodiment. Multiple sensors are preferably disposed in the housing around the peripheral edge of the output window.

The plurality of sensors preferably comprises third and fourth sensors disposed in the housing on diametrically opposing third and fourth sides of the housing output window.

The control system is preferably configured to cause one or more of the sensors to be deactivated when the head is in the engaged configuration. It will be appreciated that preferably the third and fourth sensors are deactivated when the head is in the engaged configuration.

The shield portion preferably further shields one or more of the sensors in the engaged configuration. Accordingly, in the engaged configuration the third and fourth sensors are preferably hidden by the shield portion.

The one or more sensors shielded by the shield portion are preferably proximity sensors (preferably capacitive sensors), and the shield portion is positioned adjacent to the one or more shielded proximity sensors. The shield portion may be sufficiently close to the proximity sensors in the engaged configuration such that it is not essential to deactivate third and fourth sensors. Alternatively, the proximity sensors may be functional and thereby output a signal to the control system indicating proximity to a surface. The control system is beneficially arranged to control operation of the device such as whether the light source can emit an energy pulse in part dependent on the proximity sensors indicating a predetermined proximity to a surface, meaning in the engaged configuration the proximity sensors output a positive determination of surface proximity to the control system. The one or more shielded sensors are preferably the third and fourth sensors.

The head preferably does not include any sensors. This means that the head is simple to manufacture as does not require complex electronics. It also means that the robustness of what is a relatively small attachment is increased.

The head preferably magnetically couples to the housing.

The device preferably comprises an engagement sensor arrangement for determining whether the head is in the engaged configuration. The control system may be operable to modify an operational parameter of the device dependent upon the engagement sensor arrangement output. The operational parameter may be one or more of: a) one or more of the sensors to be deactivated when the head is in the engaged configuration; b) frequency of light energy pulse emission; c) energy value of the light energy pulses emitted. The engagement sensor arrangement may comprise one or more Hall effect sensors.

The device is preferably an Intense Pulsed Light (IPL) device.

According to a further aspect of the present invention there is a skin treatment device comprising:
a housing;
a light source housed within the housing for discharging light energy pulses;
a control system for controlling discharge of the light source;
the housing having a head portion comprising a recessed zone defined between opposing shoulders for receipt of a user body portion, with an output window in the recessed zone for transmission of the light energy pulses emitted by the light source to external of the housing onto a skin treatment area, the output window having a width and a height, the width defined by first and second ends and extending in a direction between the opposing shoulders;
one or more sensors disposed in the housing for sensing a user body portion on opposing sides of the output window where the one or more sensors do not extend width wise beyond the first and second ends of the output window;
where the control system is arranged to receive sensor outputs from the one or more sensors and based on the sensor outputs control operation of the device.

Controlling operation of the device may comprise one or both of determining whether or not the flashlamp can emit a pulse and determining properties of that pulse (e.g. fluence).

The head portion is preferably integrated with the housing. This means that the head portion is preferably not detachable from the housing.

The shape of the head portion is preferably the same as described with respect to the first aspect of the invention. Preferably, the recessed portion is concave, and wherein the curvature of the concave recessed portion is defined at least in part by a curvature radius. It will be appreciated that a curvature radius requires that with a cylinder positioned into the recess there are multiple contact points between the cylinder and the contact surface of the recessed zone.

The curvature radius may comprise a numerical value defined by the width of the output window plus a predetermined value, wherein the predetermined value may be 40 mm. Accordingly, in an illustrative embodiment the width of the output window may be 10 mm, and the curvature radius may be 50 mm.

The one or more sensors are preferably disposed adjacent the output window. Preferably a first and second sensor are disposed in the housing. The first and second sensor are preferably aligned on opposing sides of the output window. The first and second sensors are preferably provided at the lowermost part of the recess. It will be appreciated that the recessed zone comprises a user contact surface.

The head is preferable rigid. This means that in normal operation the head does not deflect.

### BRIEF DESCRIPTION OF THE DRAWINGS

Aspects of the present invention will now be described by way of example only with reference to the accompanying figures where;

FIGS. 1a-c show a schematic representation of an illustrative embodiment of the present invention without a head in position;

FIG. 2 is schematic illustrative embodiment of the present invention in perspective, plan and side view respectively of the present invention, where a head (100) is secured to the front end of the housing (50);

FIG. 3 is a schematic representation of an illustrative embodiment of the present invention demonstrating an illustrative curvature of a head;

FIG. 4 is a rearward view of a head according to an illustrative embodiment of the present invention; and

FIG. 5a-d is a schematic upper perspective, front, plan and bottom perspective representation of an illustrative embodiment of the present invention.

### DETAILED DESCRIPTION OF THE INVENTION

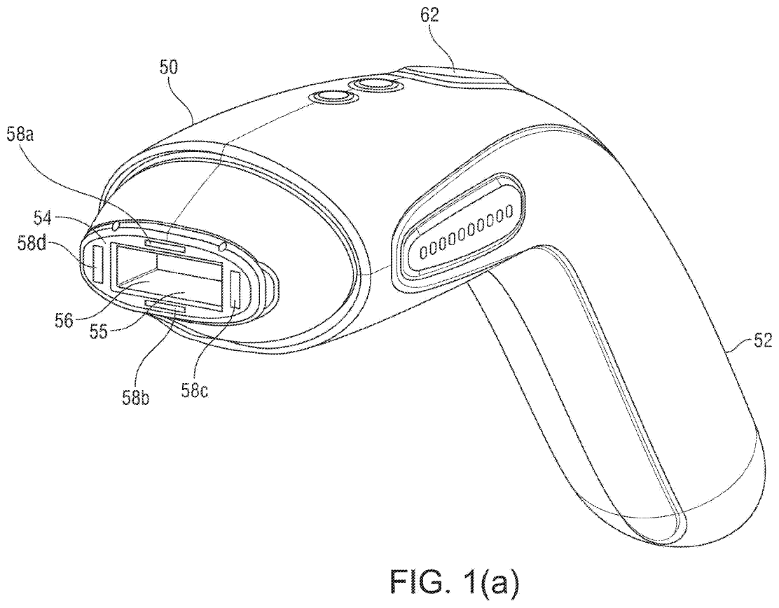

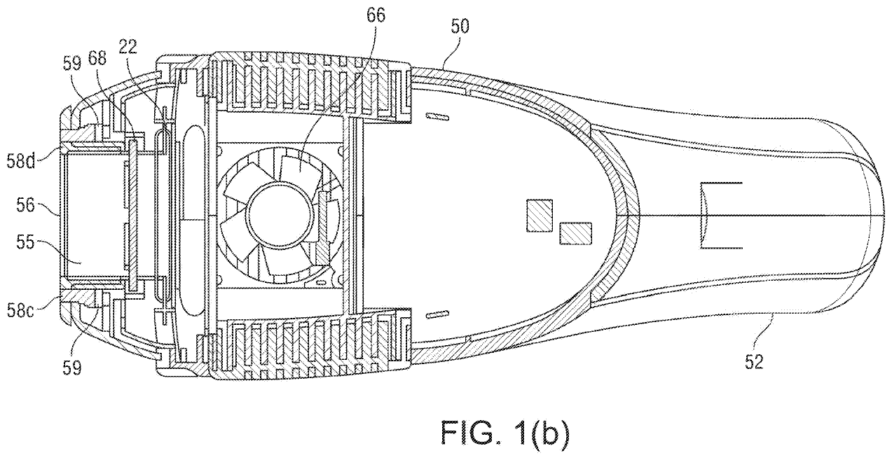

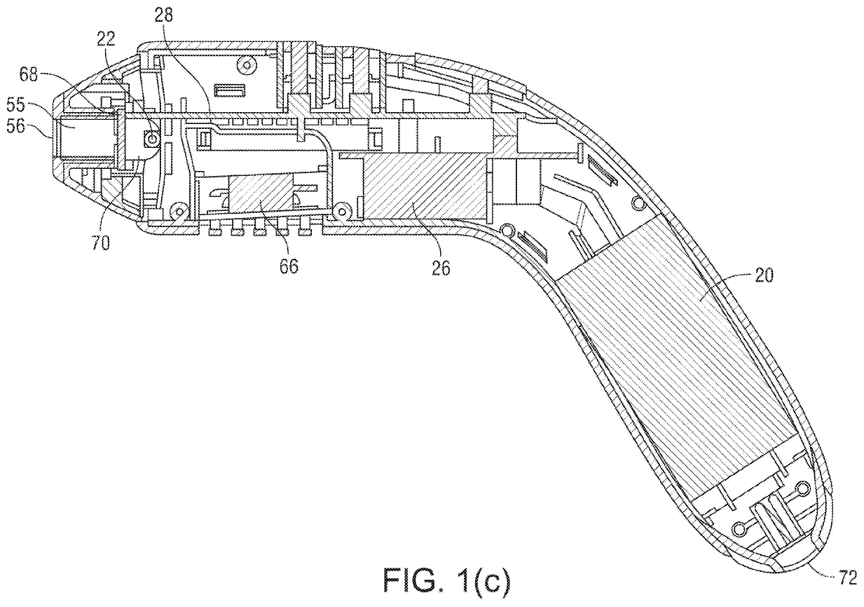

Referring to FIG. 1a-c presented is a skin treatment apparatus that may be used for treating skin disorders and conditions, and even more beneficially is suitable for cosmetic purposes such as hair depilation. The device comprises a housing (50) and a light source (22) accommodated by the housing such as a discharge lamp or flashlamp. The flashlamp is arranged to generate high intensity pulses of optical radiation. The housing (50) comprises a handle (52) meaning that the housing (50) can be manipulated to be positioned appropriately on a user and is particularly suitable for the home market as the device is handheld, relatively small and portable. The housing (50) includes a skin contact element (54) arranged in use without the provision of the head to be positioned adjacent or preferably on a user's skin. The skin contact element (54) includes a light output window (56) or transmission window to enable the passage of high intensity pulses of optical radiation therethrough typically measuring 30 mm in width and 10 mm in height, where there is a light guide (55) defined between the light output aperture/transmission window (56) and the light emitting element (22). The cross-sectional area of the light output aperture/transmission/output window (56) is effectively the treatment area. The provision of the light guide (55), which may be termed a light pipe, is that it directs the light out of the light output aperture/transmission window (56). Reflectors (not shown) are preferably included defining at least part of the walls of the light guide (55) to aid in reflection of light through the light output aperture/transmission window (56). The light emitting element (22) is accordingly recessed relative to the light output aperture/transmission window (56). The effect of provision of the light guide (55) is to improve safety through reducing the divergence of the light exiting the apparatus to the skin.

The skin contact element (54) further includes first, second, third and fourth sensors (58a, 58b, 58c, 58d) which will be described further below for providing associated sensing zones. An actuator (62), for example in the form of a push button is provided for the user to cause release of energy from the charge storage device such as a capacitor (20) to cause a pulse of optical radiation to the emitted from the flashlamp (22).

Referring to FIG. 1b, a transverse cross-section of the housing (50) is presented again showing the handle (52), light output aperture (56) and sensors (58c, 58d). Further shown is a fan (66) for cooling of the control circuit (28) on the main printed circuit board. FIG. 1b shows the lamp (22) secured in the housing (50). A filter (68) is provided to filter out ultra-violet light from transferring from the lamp (22) to the skin. A treatment light pulse generated by the lamp (22) passes through the filter, through the light output window (56) and onto the skin of a user.

Referring in particular to FIG. 1c, a cross-sectional view is taken on an axis substantially perpendicular to the view of FIG. 1b. Represented in FIG. 1c is the charger circuit (26), control circuit (28) mounted onto a printed circuit board, lamp (22), filter (68) and light output window (56). Further shown is a reflector (70) for reflecting the pulse of optical radiation and accommodated within the handle portion (52) of the housing (50) is the energy storage device comprising a capacitor (20). The handle defines an opening (72) for mains power input.

The apparatus effectively functions by the user providing an input to actuator (62) following which a determination is made as to whether a threshold response is received from all of the sensors (58) and assuming each sensor provides a threshold response then the capacitor (20) discharges over the flashlamp (22)

The sensors may take different forms dependent upon the device in which it is utilised. For example, the sensors may simply comprise multiple proximity sensors in the form of capitative proximity/contact sensors each having a sensing zone where the control system requires a predetermined capacitance to be measured from each sensing zone which is indicative of contact with a user's skin. Assuming a threshold value is measured, then the control system enables firing of the flashlamp to emit a light energy pulse. However, one or more alternative or additional skin parameters may be sensed. For example, one or more sensors may comprise an optical sensor often referred to as a skin tone sensor or sometimes again a proximity sensor and can be used in the alternative to or in tandem with one or more other sensor types such as capacitive sensors. In the embodiment presented, there are three capacitive proximity sensors and one optical proximity sensors (or ‘skin tone sensor’ (58a)). A skin tone sensor includes a transmitter arranged to transmit sensing radiation through the sensor window onto the skin to be treated. The sensor (58a) further includes a receiver such as a photodiode arranged to receive radiation reflected from a skin surface. Intensity of the received radiation is found to be representative of the tone of the skin, for example a light skin tone will reflect more than a dark skin tone. The intensity of the received radiation can be processed by the control circuit (28) using a processor provided thereby and compares the intensity with a calibrated set of intensity measurements to determine a sensed skin tone, which is then stored in a memory of the control circuit. The treatment light pulse energy then outputted to the skin can be controlled and is thus dependent on the sensed skin tone thus ensuring optimised treatment for the specific skin tone to be treated.

It will be appreciated that it is possible to utilise a single sensor having multiple sensing zones adjacent the output window (56). A single sensor may for example extend around the entirety of the output window (56), with sensing zones above, below and to either side of the output window (56). It is preferable however to provide multiple sensors adjacent the output window (56).

As indicated above, in an illustrative embodiment multiple individual sensors are disposed around the output window (56). There are typically four, the first (58a) positioned above, second (58b) positioned below and third (58c) and fourth (58d) on opposing sides of the output window (56) providing four individual sensing zones. It will be appreciated that in alternative embodiments there are different numbers of sensors. For example, a single sensor may comprise multiple sensing zones.

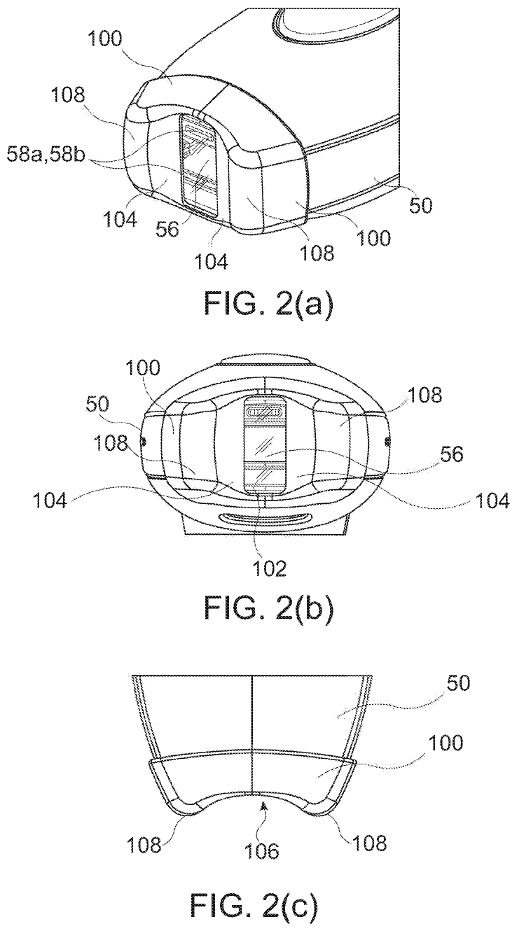

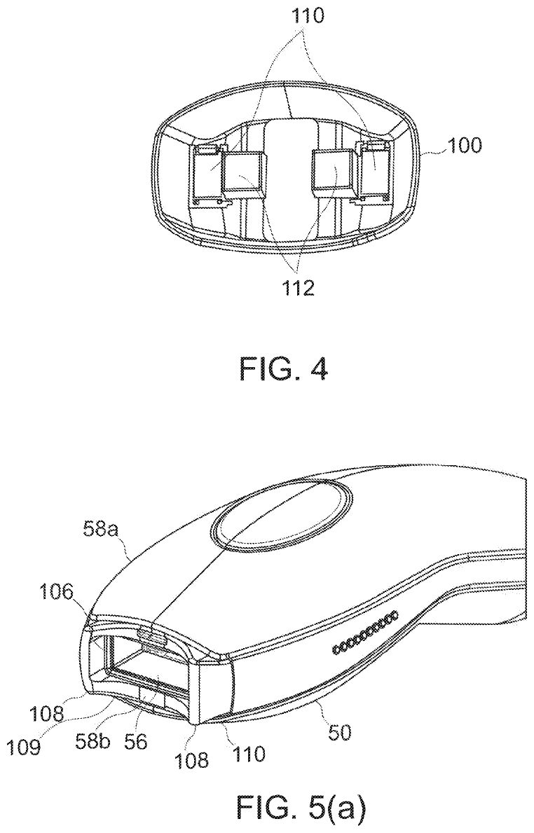

Referring now to FIG. 2 there is schematic illustrative embodiment in perspective, plan and side view respectively of the present invention, where a head (100) is secured to the front end of the housing (50). The head (50) receives the front end of the housing (50) and may be secured relative to the housing through one or more magnets as best shown in FIG. 4.

The head (100) comprises a window portion (102) and shield portion (104). Through the window portion (102) the output window (56) and first and second sensors (58a, 58b) remain exposed, whereas third and fourth sensors (58c, 58d) are shielded. The effective width of the output window (56) of the housing (50) is reduced, and in an illustration only may be reduced from 30 mm to 10 mm in width. The height remains the same such that the first and second sensors (58a, 58b) remain exposed.

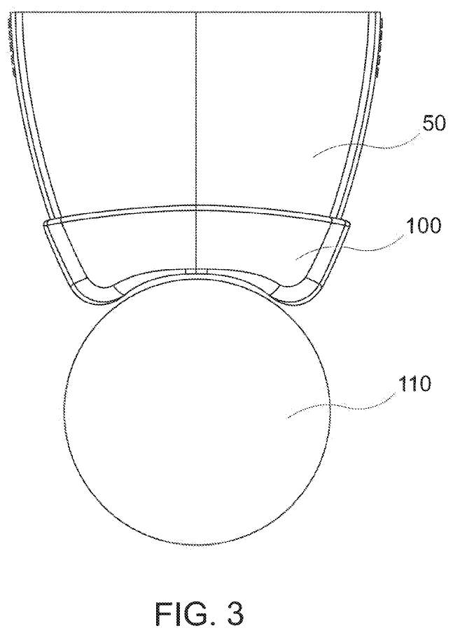

The head (100) further comprises a recess (106) defined between opposing shoulders (108) and the recess shape is concave. This shape effectively receives the user body portion that is small and/or highly curved (such as shin, arms, fingers). The curvature of the concave recess (106) is defined by a radius of curvature as shown schematically in FIG. 3, where a cylinder of radius 50 mm with a head output window (102) of width 10 mm is shown. The curvature is such that a cylinder (110) of radius 50 mm seats into the curvature of the radius of the head, at least in portions extending from the shoulders (108) toward the window portion (102). In the event that the head output window was increased to a width of 20 mm, then the radius of curvature of the concave recess would be 60 mm. This determination is made to minimise stray radiation escaping from the head output window.

The opposing shoulders (108) are mirrored on opposing sides of the recess (106) and extend generally longitudinally and are generally extend parallel to the height of the output window (56). It will be appreciated for the device to operate, the first and second sensors (58a, 58b) must receive an input signal indicative of proximity to a user's skin, and together with the recess (106) stray light is minimised.

It is an optional feature that the control system causes deactivation of the third and fourth sensors (58c, 58d) when the head (100) is engaged with the housing (50). For this to occur, the control system must receive an input indicating that the head (100) is engaged. Referring to FIG. 4, a rearward portion of the head (100) is presented. In the head (100) are a pair of magnets (110) for enabling magnetic coupling with the housing (50). The housing (50) may include corresponding metallic elements appropriately aligned with the magnets (110) to ensure coupling that is sufficiently secure such that the head will not decouple during normal use. Further provided is an engagement sensor arrangement such as one or more Hall effect sensors (not shown) that is/are capable of detecting the presence of the head (100) in the engaged configuration via the magnetic field from the magnets. The Hall effect sensor(s) can then provide an output to the control system indicating engagement with the head (100) and cause sensors (58c, 58d) to be deactivated, thereby requiring only a threshold signal from the sensors (58a, 58b) to allow emission of a light energy dose.

The engagement sensor arrangement may have further functionality in identification of a particular head that is engaged. Multiple head sizes may be provided for treatment of different body areas each having different user contact surface configurations where, for example, the radius curvature is different. The engagement sensor arrangement may be arranged to determine which head is engaged (for example through multiple Hall effect sensors and different magnet configuration for each head size) and based on this information control an output parameter accordingly.

In an embodiment of the invention a single sensor may be provided, typically but not essentially extending around the entire periphery of the output window (56), where with the head (100) engaged only certain sensing zones remain exposed. For example, sensing zones above and below the output window may remain exposed, and sensing zones to either side of the output window may be shielded. In this embodiment, with the head attached the threshold level for the sensor output may be modified to a lower value to compensate for the fact that the sensing zones to either side of the output window cannot determine proximity of skin as they are shielded. This can be automated by determination by the control system that the head is in the engaged position by virtue of the output from the engagement sensor arrangement.

Further shown in FIG. 4 are the provision of reflector shields (112). These reflector shields (112) provide the shield portion (104) of the head (100) and thus reduce the skin transmission window of the housing and accordingly the skin treatment area. These shields (112) are reflective and insulating such that the head (100) does not absorb significant amounts of energy and heat up excessively.

The provision of the engagement sensor arrangement may have additional beneficial uses. For example, the output from the engagement sensor arrangement may cause the control system to modify an operational parameter of the device, where the operational parameter may be the pulse emission rate from the light source, the energy output from each pulse, and/or as described above the operation state of one or more of the sensors. The pulse emission rate may be modified to reduce the possibility of the head overheating, so the control system may reduce the rate automatically when the head (100) is in the engaged configuration. Furthermore, the energy output from the light source may be modified to maintain the same fluence (energy per unit area) on the skin as if the head (100) was not in place.

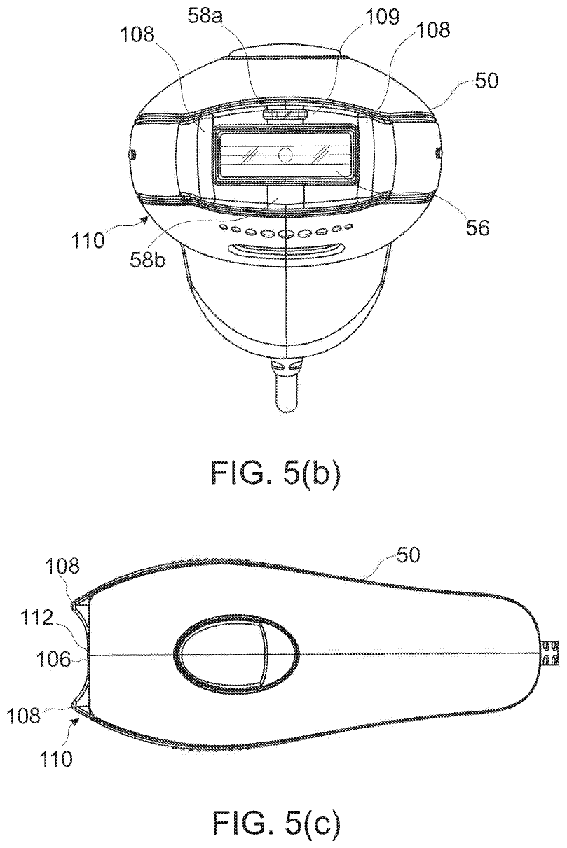

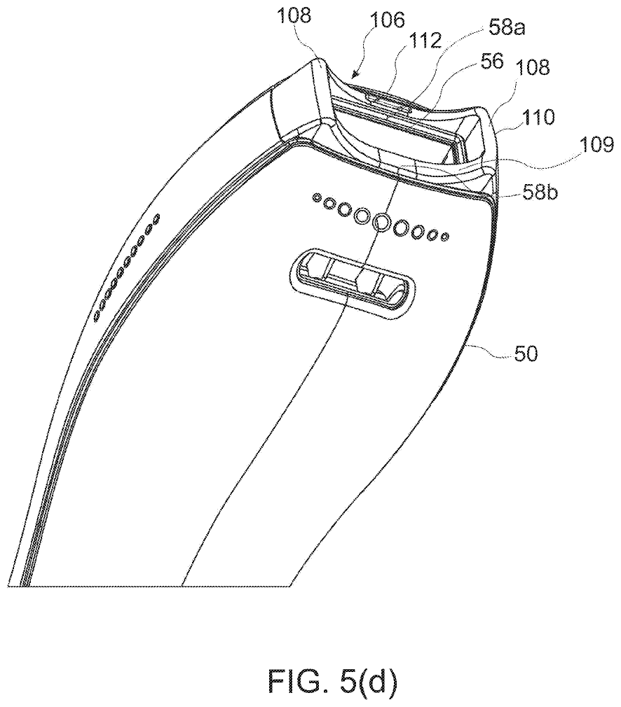

Referring now to FIG. 5, there is a schematic perspective representation of an embodiment according to a second aspect of the present invention wherein the housing (50) includes an integrated head portion where the head portion has the same recess (106) geometry as the first embodiment above providing a recessed zone for receipt of a user body portion. FIG. 5a is a schematic upper perspective view. FIG. 5b a front view, FIG. 5c a plan view and FIG. 5d a bottom perspective representation of such an illustrative embodiment of the present invention.

In this embodiment the head portion (110) is not detachable, and the area of the light output window (56) and thus the treatment area remain unmodified. Components analogous to the first embodiment have been referenced with the same reference numerals. The functionality of the device may be the same as that of the first embodiment as described with respect to FIG. 1, with the differences being the head portion (110).

The head portion (110) comprises a recessed zone (106) defined between opposing shoulders (108) and the recess shape is concave. This shape comprises a skin contact portion (109) in the form of a rim. Accordingly, the recessed zone (106) effectively receives the user body portion that is small and/or highly curved (such as shin, arms, fingers). The curvature of the skin contact portion (109) of the concave recess (106) is defined at least in part by a radius of curvature and this can be demonstrated in the same way as shown in FIG. 3. In the illustrative embodiment of FIG. 5, the output window (56) has a width of 30 mm, and the radius or curvature is therefore beneficially 70 mm. It will be appreciated that as clearly shown in the Figures the curvature radius does not extend the full distance between the opposing shoulders (108). Instead, the curvature radius extends inwardly from the shoulders (108) towards an intermediate location (112) of the skin contact portion (109), where the rate of curvature decreases towards the intermediate location (112).

The opposing shoulders (108) are mirrored on opposing sides of the recess (106) and extend generally longitudinally and are generally extend parallel to the height of the output window (56). It will be appreciated for the device to operate, the first and second sensors (58a, 58b) must receive an input signal indicative of proximity to a user's skin, and together with the recess (106) stray light is minimised. The first and second sensors (58a, 58b) are provided in the intermediate location (112) of the skin contact portion (109) on opposing sides of the output window (56). These sensors do not extend width wise beyond the first and second ends of the output window (56). In fact, the output window (56) extends adjacent the shoulders (108).

Functionality of the device as presented in FIG. 5 is significantly increased due to the head portion shape and sensor positioning for curved areas of the body, whilst safety is ensured by minimising the emission of stray light energy. Due to the recessed zone of the head portion, it is not a requirement to provide sensors to the sides of the output window, and by not providing such sensors usability is increased as it is only necessary for sensors above and below the output window to determine the presence of skin for the device to emit an energy pulse, however safety is not compromised as the curvature ensures stray light is minimised. Furthermore, the device complexity is reduced as less sensors or sensing zones are required.

Aspects of the present invention have been described by way of example only and it will be appreciated by the skilled addressee that modifications and variations may be made without departing from the scope of protection afforded by the appended claims.

1. A skin treatment device comprising:
a housing;
a light source housed within the housing for discharging light energy pulses;
a control system for controlling discharge of the light source;
a housing output window in the housing for transmission of the light energy pulses emitted by the light source to external of the housing onto a skin treatment area;
one or more sensors disposed in the housing adjacent the housing output window to provide a plurality of sensing zones, where the control system is arranged to receive one or more sensor outputs from the one or more sensors and based on the one or more sensor outputs control operation of the device;
a head arranged to be releasably engaged with the housing and having a shield portion and a head window portion, where in an engaged configuration the shield portion partially shields the output window to reduce the skin treatment area and leaves one or more of the plurality of sensing zones exposed.

2. A skin treatment device according to claim 1 wherein the head comprises a recessed portion arranged to receive a user body portion.

3. A skin treatment device according to claim 2 wherein the recessed portion is concave.

4. A skin treatment device according to claim 2 wherein the recessed portion is disposed between opposing shoulders.

5. A device according to claim 1 wherein the curvature of the concave recessed portion is defined at least in part by a curvature radius.

6. A skin treatment device according to claim 5 wherein the curvature radius has a numerical value defined by the width of the head window as shielded plus a predetermined value, wherein the predetermined value is 20-60 mm.

7. A skin treatment device according to claim 1 wherein the head is rigid.

8. A skin treatment device according to claim 1 wherein the window portion of the head comprises an opening.

9. A skin treatment device according to claim 1 wherein the housing output window is defined by a width and a height, where the width is greater than the height and where the shield portion shields the output window to reduce the width of the light transmission area in the engaged configuration.

10. A skin treatment device according claim 9 where the head output window is defined by a width and a height aligned with the width and height respectively of the housing output window in the engaged configuration, where the width of the head output window is less than the height of the head output window.

11. A skin treatment apparatus according to claim 9 wherein the height of the head output window is greater than the height of the housing output window.

12. A skin treatment apparatus according to any claim 1 wherein the one or more sensors comprises at least first and second sensors disposed in the housing on diametrically opposing first and second sides of the housing output window, and where the first and second sensors remain exposed in the engaged configuration.

13. A skin treatment device according to claim 11 wherein the plurality of sensors comprises third and fourth sensors disposed in the housing on diametrically opposing third and fourth sides of the housing output window.

14. A skin treatment device according to claim 1 wherein the control system is configured to cause one or more of the sensors to be deactivated when the head is in the engaged configuration.

15. A skin treatment device according to claim 1 wherein the shield portion further shields one or more of the sensors in the engaged configuration.

16. A skin treatment device according to claim 13 wherein the one or more sensors shielded by the shield portion are proximity sensors, and the shield portion is positioned adjacent to the one or more shielded proximity sensors.

17. A skin treatment device according to claim 1 wherein the head does not include any sensors.

18. A skin treatment device according to claim 1 wherein the head magnetically couples to the housing.

19. A skin treatment device according to claim 1 further comprising an engagement sensor arrangement for determining whether the head is in the engaged configuration, and wherein the control system is operable to modify an operational parameter of the device dependent upon the engagement sensor arrangement output.

20. A skin treatment device according to claim 1 wherein the device is an Intense Pulsed Light (IPL) device.

21. A skin treatment device according to claim 1 operable both with and without the head engaged with the housing.

22. A skin treatment device comprising:
a housing;
a light source housed within the housing for discharging light energy pulses;
a control system for controlling discharge of the light source;
the housing having a head portion comprising a recessed zone defined between opposing shoulders for receipt of a user body portion, with an output window in the recessed zone for transmission of the light energy pulses emitted by the light source to external of the housing onto a skin treatment area, the output window having a width and a height, the width defined by first and second ends and extending in a direction between the opposing shoulders;
one or more sensors disposed in the housing for sensing a user body portion on opposing sides of the output window where the one or more sensors do not extend width wise beyond the first and second ends of the output window;
where the control system is arranged to receive sensor outputs from the one or more sensors and based on the sensor outputs control operation of the device.

23. A skin treatment apparatus according to claim 22 wherein the head portion is integrated with the housing.

24. A skin treatment device according to claim 23 wherein the recessed portion is concave.

25. A skin treatment device according to claim 24 wherein the curvature of the concave recessed portion is defined at least in part by a curvature radius.

26. A skin treatment device according to claim 25 wherein the curvature radius has a numerical value defined by the width of the output window plus a predetermined value, wherein the predetermined value may be 40 mm.
